SEQUENCE LISTING (4 sequences)

SEQ ID NO: 1 - DNA, 21 bp, Homo sapiens
gatatttgatsgtggtccttt

SEQ ID NO: 2 - DNA, 21 bp, Homo sapiens
ctgtgaatttwgtgatgcact

SEQ ID NO: 3 - DNA, 21 bp, Homo sapiens
gattgtatccygatggaaatg

SEQ ID NO: 4 - DNA, 21 bp, Homo sapiens
aagtccccaastgcaaagctc

(19) United States
(12) Patent Application Publication
(10) Pub. No.: US 2012/0040342 A1
(43) Pub. Date: Feb. 16, 2012

(54) Genetic Indicators Of Weight Loss

(75) Inventors: Glenn Gerhard, Lewisburg, PA (US); Christopher Doubet Still, Winfield, PA (US); Peter N Benotti, Fort Lee, NJ (US); Xin Chu, Lewisburg, PA (US)

(73) Assignee: GEISINGER CLINIC, Danville, PA (US)

(21) Appl. No.: 12/933,002

(22) PCT Filed: Mar. 17, 2009
(86) PCT No.: PCT/US2009/037401

§ 371 Date: Jan. 18, 2011

Related U.S. Application Data
(60) Provisional application No. 61/037,173, filed on Mar. 17, 2008.

(51) Int. Cl. C12Q 1/68

(52) U.S. Cl. 435/6.11

(57) ABSTRACT

Methods for determining resistance to weight loss and susceptibility to binge eating episodes are described. The methods include determination of the presence of a obesity related alleles for a patient at single nucleotide polymorphism sites associated with the genes INSIG2, FTO, MC4R, and PCSK1. The total number of obesity alleles for the patient is indicative of the patient's resistance to weight loss and susceptibility to weight gain following bariatric surgery. The methods also include determining if a patient is homozygous for an obesity related allele at one or more single nucleotide polymorphism sites of the four genes.

### STATEMENT OF PRIORITY

This application claims priority to U.S. Provisional Application No. 61/037,173, filed Mar. 17, 2008, whose disclosure is hereby incorporated by reference herein.

### FIELD OF THE INVENTION

The present invention relates to genetic predictors of weight, particularly single nucleotide polymorphisms associated with weigh loss outcomes.

### BACKGROUND OF THE INVENTION

Obesity, commonly defined as a body mass index (BMI) greater than 30 kg/m², is directly related to an increased risk for diabetes mellitus, hypertension, dyslipidemia, cardiovascular disease, certain forms of cancer, and to overall mortality⁷. Morbid obesity (BMI>40 kg/m²) further increases disease burden and risk of mortality^{8,9}. Weight loss is effective at decreasing these risks and ameliorating disease severity¹⁰, thus reducing body weight is a major clinical goal. Currently available dietary and pharmacological modalities may produce small to moderate levels of weight loss, but in most patients are either not achieved or are not sustained⁴.

Bariatric surgery has thus emerged as a highly effective therapy for long-term weight loss in morbidly obese patients¹, and more recently as a surgical therapy for the potential cure of type 2 diabetes^{2,3}. However, the degree of weight loss and treatment success is variable⁵. A major clinical need for the management of obesity is thus the ability to stratify patients into specific therapeutic modalities, which has not yet been met by available clinical and demographic variables¹¹.

One factor that may influence a patient's risk for obesity, and therefore the potential long-term success of bariatric surgery, is genetic susceptibility. Twin and adoption studies support an important role for genetic factors influencing the development of obesity.⁴⁷ However, most cases of adult obesity are not caused by single genetic defects.⁴⁸ Efforts have therefore focused on the identification of genetic variants that predispose carriers to common, polygenic obesity. A large number of common genetic variants have been reported to be related to BMI, but few of the associations have been reproduced across multiple populations.¹³ Most studies have also been performed in individuals with normal weight, overweight, and class I obesity and have not included morbidly obese patients.

Bariatric surgery, while an effective weight loss option for many patients, has underlying risks. As such, information regarding a patient's genetic predisposition to weight loss may assist the physician and patient in determining the type of bariatric surgery best suited to the patient, or even whether to undergo bariatric surgery at all. Therefore, there exists a need for methods which are able to accurately determine which patients may be more resistant to weight loss.

### SUMMARY OF THE INVENTION

In one aspect of the present invention, methods are provided for determining a patient's resistance to weight loss or likelihood to achieve weight loss (e.g. a predisposition to changes in body mass index). The methods involve determining the presence of certain obesity alleles at single nucleotide polymorphism (SNP) positions.

The SNPs of the present invention include SNPs associated with the human genes INSIG2 (rs7566605), FTO (rs9939609), MC4R (rs17782313) and PCSK1 (rs6235). The obesity related alleles are C for the rNSIG2 SNP, A for the FTO SNP, C for the MC4R SNP and C for the PCSK1 SNP.

In one aspect of the present invention, methods are provided for determining a patient's predisposition to changes in BMI by totaling the number of obesity alleles for the patient at each of the INSIG2 SNP, FTO SNP, MC4R SNP and PCSK1 SNP. If the total number of obesity alleles is between 5-8, it is indicated that the patient is resistant to weight loss.

In another aspect of the present invention, methods are provided for determining a patient's predisposition to changes in BMI by totaling the number of homozygous obese genotypes for the patient for each of the INSIG2 SNP, FTO SNP, MC4R SNP and PCSK1 SNP. The presence of two of more homozygous obese genotypes are indicative that the patient is resistive to weight loss.

In another aspect of the present invention, methods are provided for determining a patient's predisposition to changes in BMI by analysis of three of fewer of the INSIG2 SNP, FTO SNP, MC4R SNP and PCSK1 SNP.

The present invention can be used for informing physician decisions regarding the form of treatment of obese patients. If the methods of the present invention indicate a resistance to weight loss, more invasive or dramatic procedures may be required. Alternatively, if the methods of the present invention indicate a susceptibility to weight loss, bariatric surgery or other treatments may be indicated as desirable.

In a still further aspect of the present invention, methods are provided for determining a patient's susceptibility to binge eating episodes. If a patient is found to be homozygous for the obesity allele of the INSIG2 SNP, the patient is indicated as being susceptible to binge eating episodes.

In yet a further aspect of the present invention, methods are provided for determining a patient's metabolic rate or resting energy expenditure or oxygen consumption (VO₂). If a patient is found to be homozygous for the obesity allele of the INSIG2 SNP, the patient is indicated as being susceptible to having a lower metabolic rate or resting energy expenditure or oxygen consumption (VO₂).

### DETAILED DESCRIPTION OF THE DRAWINGS

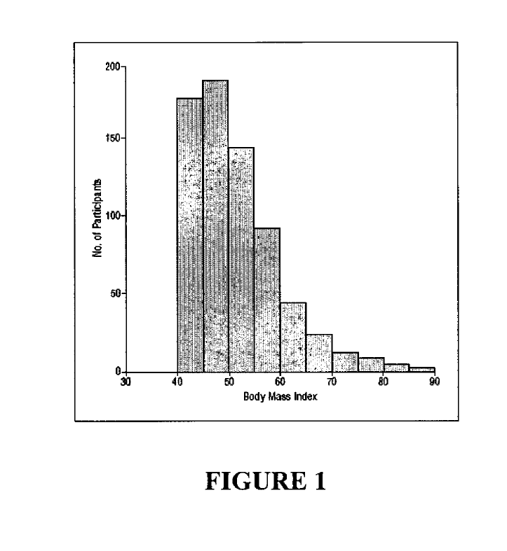

FIG. 1 shows a histogram of BMI (calculated as weight in kilograms divided by height in meters squared) in morbidly obese patients.

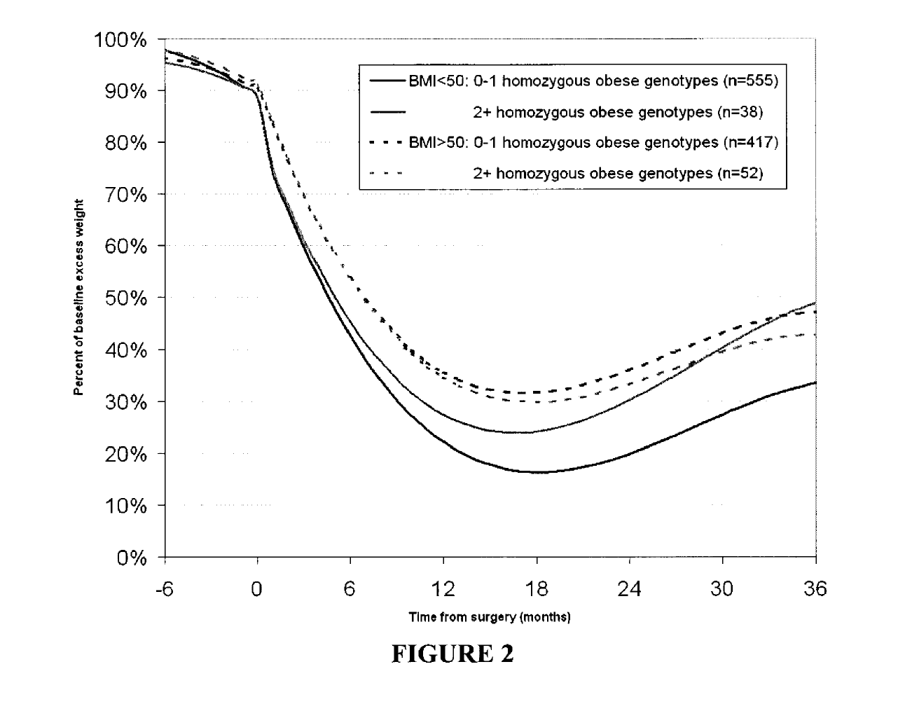

FIG. 2 shows a plot of the percent of baseline excess weight vs. time from bariatric surgery in months for patients with a starting BMI of less than 50 (solid lines) and a starting BMI of greater than 50 (dashed lines). The plot shows the differences in post-operative weight changes between patients having 0-1 homozygous obese genotypes for the INSIG2, FTO, MC4R and PCSK1 SNPs (black lines) and patients having 2 or more obese genotypes for the same SNPs (gray lines).

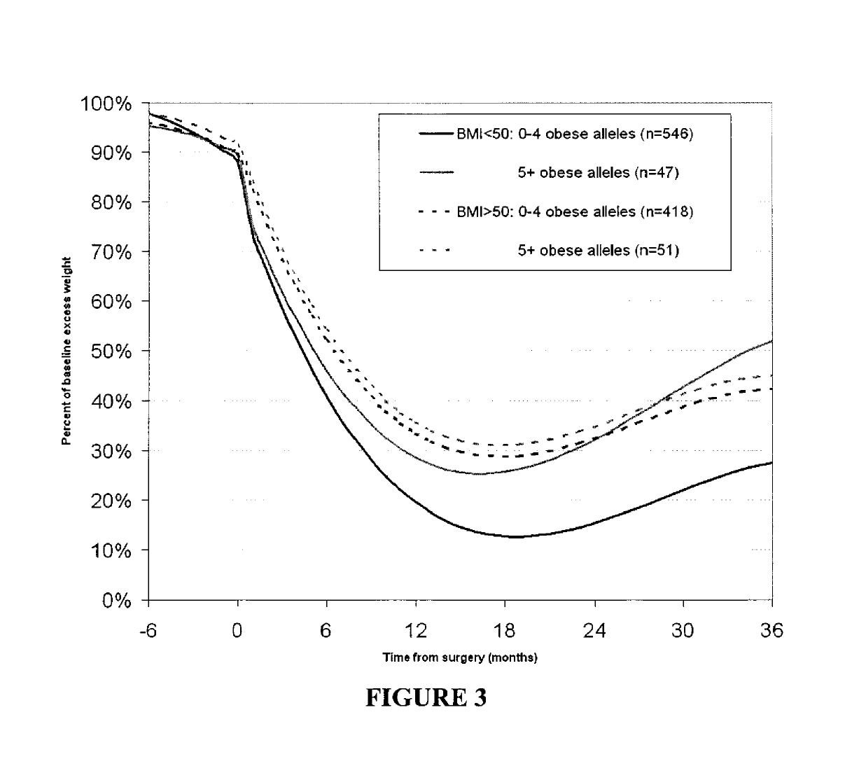

FIG. 3 shows a plot of the percent of baseline excess weight vs. time from bariatric surgery in months for patients with a starting BMI of less than 50 (solid lines) and a starting BMI of greater than 50 (dashed lines). The plot shows the differences in post-operative weight changes between patients having 0-4 obese alleles for the INSIG2, FTO, MC4R and PCSK1 SNPs (black lines) and patients having 5 or more obese genotypes for the same SNPs (gray lines).

### DETAILED DESCRIPTION OF THE INVENTION

The present invention provides methods for determining a person's susceptibility to obesity and resistance to weight loss. The present invention provides methods for analysis of genetic factors associated with obesity. Particularly, the present invention provides methods for analyzing specific single nucleotide polymorphisms (SNPs), which are associated with obesity and resistance to weight loss.

The present invention provides methods for determining the presence of a specific allele for one or more SNP. The presence of a specific allele is then correlated with a patient's likelihood to be resistant to weight loss or their likelihood to achieve significant weight loss. In embodiments of the invention, the methods can be performed using a single SNP, or multiple SNPs, e.g. two SNPs, three SNPs or four SNPs, as are described herein below.

In a specific embodiment of the present invention, SNPs that occur naturally in the human genome are provided as isolated nucleic acid molecules. In particular the SNPs are associated with weight loss outcomes. As such, they can have a variety of uses in the diagnosis and/or treatment of obesity and related pathologies. One aspect of the present invention relates to an isolated nucleic acid molecule comprising a nucleotide sequence in which at least one nucleotide is a SNP. In an alternative embodiment, a nucleic acid of the invention is an amplified polynucleotide, which is produced by amplification of a SNP-containing nucleic acid template. In another embodiment, the invention provides for a variant protein that is encoded by a nucleic acid molecule containing a SNP disclosed herein.

The specification uses designations of nucleic acid residues well know in the art, using standard abbreviations: e.g. adenosine (A), guanosine (G), thymidine (T) and cytidine (C). For example, the indication that a residue C is present at a specific position is an indication that a cytidine residue is present at that position. Further, standard abbreviations are used in the sequence listing for positions that have two possible residues: with G or C represented by S; A or T represented by W; and T or C represented by Y.

One SNP of the present invention is in the region of the human gene INSIG2, on chromosome 2. The SNP has a Reference SNP Cluster ID number of rs7566605 in the National Center for Bioformation's Entrez SNP database. The INSIG2 SNP is represented by position 11 of SEQ ID NO: 1, which is the sequence surrounding the INSIG2 SNP. The obesity related allele for the INSIG2 SNP is a C at position 11 of SEQ ID NO: 1. All SNPs with significant linkage disequilibrium (D>0 or D<0; D′>0 or D′<0) with this SNP are also contemplated by the present invention. Other SNPs that are in or nearby this gene that function in a similar manner are also included.

Another SNP of the present invention is in the region of the human gene FTO, on chromosome 16. The SNP has a Reference SNP Cluster ID number of rs9939609 in the Entrez SNP database. The FTO SNP is represented by position II of SEQ ID NO: 2, which is the sequence surrounding the FTO SNP. The obesity related allele for the FTO SNP is an A at position 11 of SEQ ID NO, 2. All SNPs with significant linkage disequilibrium (D>0 or D<0; D′>0 or D′<0) with this SNP are also contemplated by the present invention. Other SNPs that are in or nearby this gene that function in a similar manner are also included.

Yet another SNP of the present invention is in the region of the human gene MC4R, on chromosome 18. The SNP has a Reference SNP Cluster ID number of rs17782313 in the Entrez SNP database. The MC4R SNP is represented by position 11 of SEQ ID NO: 3, which is the sequence surrounding the MC4R SNP. The obesity related allele for the MC4R SNP is a C at position 11 of SEQ ID NO: 3. All SNPs with significant linkage disequilibrium (D>0 or D<0; D′>0 or D′<0) with this SNP are also contemplated by the present invention. Other SNPs that are in or nearby this gene that function in a similar manner are also included.

The fourth SNP of the present invention is in the region of the human gene PCSK1, on chromosome 5. The SNP has a Reference SNP Cluster ID number of rs6235 in the Entrez SNP database. The PCSK1 SNP is represented by position 11 of SEQ ID NO: 4, which is the sequence surrounding the PCSK1 SNP. The obesity related allele for the PCSK1 SNP is a C at position 11 of SEQ ID NO: 4. All SNPs with significant linkage disequilibrium (D>0 or D<0; D′>0 or D′<0) with this SNP are also contemplated by the present invention. Other SNPs that are in or nearby this gene that function in a similar manner are also included.

In one embodiment of the present invention, the total number of obesity related alleles for each copy of the four SNPs of the present invention is determined. As there are two copies of each allele, a determination of the number of obesity alleles for the four SNPs will give a number of 0-8 obese alleles. For example, the presence of the residue C for one copy INSIG2 SNP will co-ant as one obesity related allele. The total number of obese alleles can then be correlated with a risk of obesity, resistance to weight loss (e.g. resistance to change in body mass index (BMI), likelihood of successful weight loss) and suitability for bariatric surgery. In certain embodiments of the invention, the presence of 5 or more obese alleles in a subject suggests a genetic resistance to weight loss, and subjects bearing this number of obese alleles are indicated as resistant to changes in BMI following circumstances promoting weight loss such as surgical therapies. Additionally, the presence of 4 or fewer obese alleles in a subject suggests a genetic susceptibility to weight loss, and subjects bearing this number of obese alleles are indicated as susceptible to changes in BMI following circumstances promoting weight loss such as surgical therapies. It is also contemplated that other embodiments of the invention which evaluate all four SNPs may also look for 4 or more, 6 or more, 7 or more, or the presence of 8 obese alleles in determining a correlation.

In other embodiments of the invention, the total number of obesity alleles for less than all four SNPs of the invention are analyzed in order to make a genetic determination. Only three of the SNPs may be evaluated in order to determine a number of obesity alleles from 0-6, only two of SNPs may be evaluated to determine a number between 0-4 and only one SNP may be evaluated to determine a number between 0-2. In these cases the presence of half or more of the total number of obesity alleles (e.g. 3 or more out of 6), suggests a genetic resistance to weight loss, and subjects bearing this number of obese alleles are indicated as resistant to changes in BMI following circumstances promoting weight loss such as surgical therapies. Additionally, the presence of half or fewer of the total number of obesity alleles (e.g. 3 or fewer out of 6), suggests a genetic susceptibility to weight loss, and subjects bearing this number of obese alleles are indicated as susceptible to changes in BMI following circumstances promoting weight loss such as surgical therapies. The analysis of the present invention can be done with any possible combination of the four SNPs of the invention.

In another embodiment of the present invention, the total number of homozygous obese genotypes out of the four SNPs of the invention is determined. For example, the presence of a C at both copies of the INSIG2 SNP would be counted as one homozygous obese genotype. The total number of homozygous obese genotypes can then be correlated with a risk of obesity, resistance to weight loss (e.g. resistance to change in BMI) and suitability for bariatric surgery. In certain embodiments of the invention, the presence of 2 or more homozygous obese genotypes in a subject suggests a genetic resistance to weight loss, and subjects bearing this number of obese alleles are indicated as resistant to changes in BMI following circumstances promoting weigh loss such as surgical therapies. Additionally, the presence of 1 or fewer homozygous obese genotypes in a subject suggests a genetic susceptibility to weight loss, and subjects bearing this number of obese alleles are indicated as susceptible to changes in BMI following circumstances promoting weigh loss such as surgical therapies. It is also contemplated that other embodiments of the invention which evaluate all four SNPs may also look for 1 or more, 3 or more, or the presence of 4 homozygous obese genotypes in determining a correlation.

In other embodiments of the present invention, the total number of homozygous genotypes may be determined for less than all four of the SNPs of the invention. Only three, two or one SNP may be analyzed to determine the number of homozygous obese genotypes. The number of homozygous obese genotypes can then be compared with the total number of SNPs analyzed, with the presence of one or more homozygous obese genotypes suggests a genetic resistance to weight loss, and subjects bearing this number of obese alleles are indicated as resistant to changes in BMI following circumstances promoting weigh loss such as surgical therapies. Additionally, subjects bearing no homozygous obese genotypes are indicated as susceptible to changes in BMI following circumstances promoting weigh loss such as surgical therapies.

In a still further embodiment of the invention, the presence of a homozygous obese genotype for the INSIG2 SNP can further be associated with an increased frequency of binge eating. In a manner analogous to that described above, if the INSIG2 SNP is shown to be homozygous for the obese allele, then the patient is indicated as likely to suffer from episodes of binge eating. If a patient is determined to be likely to suffer from binge eating episodes, the patient may be given counseling and education to assist the patient with avoiding binge eating episodes.

The analysis of the SNPs of the present invention can be done using any sequencing method known in the art. The sequence of the nucleic acid surrounding the SNP may be determined as is well known. For example, nucleic acids comprising all or part of SEQ ID NOs: 1-4 may be amplified from a patient sample using polymerase chain reaction (PCR). The sequences of the amplified nucleic acids may then be determined, including the presence of a specific residue at the SNP position. In order to amplify nucleic acids comprising all or part of SEQ ID NOs: 1-4, primers complementary to regions outside of the nucleic acid to be amplified must be used. Creation and use of such primers for the amplification of a region of a nucleic acid is well known to those of skill in the art. Various embodiments of the invention also provide kits comprising SNP detection reagents, and methods for detecting the SNP's disclosed herein by employing detection reagents.

Alternatively, other methods of sequencing may be used to determine the allele at the SNPs of the invention, including whole genome or single chromosome sequencing methods. Additionally, other non-sequencing methods which are capable of determining the residue at the SNP may also be used. It should be apparent to one of skill in the art that, if a patient has had part or all of his genome sequenced, the sequence information may be used to determine the presence of obesity linked alleles at the SNPs of the invention.

Nucleic acid may be obtained from various patient samples, as are well known in the art, including blood, cerebrospinal fluid, saliva and other body fluids, as well from other samples such as a buccal scrape or from a tissue sample obtained from the patient.

The methods of the present invention are useful in guiding decisions regarding bariatric procedures. The methods are applicable to all known bariatric procedures, including malabsorptive procedures, restrictive procedures and mixed procedures, as are well known in the art. In certain embodiments, the methods of the present invention can be used to guide a physician as to performing Roux-en-Y gastric bypass surgery, however, other forms or bariatric procedures are also contemplated.

After a patient's sample is analyzed and the necessary SNP alleles determined, the information obtained may be used to guide the patient's treatment. For example, patients who have between 0-4 obesity alleles from analysis of all four SNPs would be likely to respond well to bariatric surgery and, as such, are good candidates for such procedures. Alternatively, the number of obesity alleles may guide the physician towards performing a more malabsorptive bariatric procedure. For example, patients who have between 5-8 obesity alleles from analysis of all four SNPs may still be candidates for bariatric surgery, however, the patient will likely find success from a more highly malabsorptive procedure. In this case, a more malabsorptive procedure (e.g. a procedure that leaves less of the stomach and small intestine in contact with consumed food) can be done for patients having a higher number of obesity alleles.

It is also contemplated that the information obtained from the methods of the present invention may be used to guide other medical decisions related to weight loss, such as highly restrictive dieting and other measures.

The other methods of the present invention, including the analysis of homozygous obesity genotypes and analysis of one to three SNPs, can also be used to guide physician decisions related to bariatric surgery and other medical procedures.

It is further contemplated that the methods of the present invention can be used for developing databases containing information on the association between the obesity alleles of the present invention and actual clinical outcomes. The databases may include information about a specific patient's number of obesity alleles correlated with actual weight loss either by dieting, bariatric surgery, or both. Thus, as more information on a larger group of patients is gathered, continually improved predictions can be made as to the association between the obesity alleles of the invention and weight loss.

It will be apparent to those of skill in the art that there are other embodiments of the present invention not explicitly described in this specification. Further, the Examples below are informational and are not intended to limit the scope of the invention. The scope of the present invention should be interpreted according to the claims presented below.

### EXAMPLES

### Example 1

### Association of FTO and INSIG2 SNPs with BMI

As an initial step in understanding potential genetic influences in patients undergoing bariatric surgery, the association of FTO and INSIG2 SNPs with BMI was determined in a large cohort of morbidly obese patients enrolled in a bariatric surgery program. Because of the role of INSIG2 in lipid and cholesterol metabolism, the effect of the 2 obesity genes on blood-lipid parameters was also analyzed.

### Methods

A. Patients

Patients undergoing open or laparoscopic Roux-en-Y gastric bypass operations or laparoscopic adjustable gastric banding procedures for morbid obesity or its comorbid medical problems at Geisinger Medical Center, Danville, Pa., were enrolled in a clinical research program on obesity and metabolic syndrome. All patients undergoing bariatric operations at Geisinger Medical Center are required to participate in a standardized multidisciplinary preoperative Program, which includes obtaining standardized clinical and laboratory data at designated times. An accurately measured BMI is obtained at the first visit at the weight management clinic. Blood samples for DNA and lipid measures were obtained approximately 3 weeks before the date of operation. Total cholesterol, high-density lipoprotein cholesterol, low-density lipoprotein cholesterol (calculated), triglyceride levels, and the cholesterol to high-density lipoprotein ratio were also measured using standard clinical laboratory techniques. The institutional review board at the Geisinger Medical Clinic approved the research protocol and all participants provided written informed consent.

B. Data Acquisition

Demographic, BMI, and laboratory data were obtained through an electronic search of EpicCare electronic medical records (Epic Systems, Verona, Wis.). The electronic medical record data were imported into SAS/STAT software (SAS Institute Inc, Cary, N.C.) and were mapped to predefined fields. The resulting data were available for statistical analysis in SAS or for export into other software applications.

C. DNA Isolation

DNA was extracted from 0.35 mL of EDTA-anticoagulated whole blood using the Qiagen MagAttract DNA Blood Midi M48 Kit and Qiagen BioRobot M48 Workstation (Qiagen, Valencia, Calif.) according to the manufacturer's directions. The final elution volume was 200 μL. For a few patients, blood was not available, so DNA was extracted from fixed liver tissue. Livers were first treated with proteinase K (1 μg/μL) in 350 μL of Qiagen Tissue Lysis Buffer (Qiagen) and incubated at 55° C. overnight. Following digestion, samples were loaded onto the Qiagen BioRobot M48 Workstation and DNA was extracted, as described for blood samples. Quantification of extracted DNA was performed using a NanoDrop ND-1000 spectrophotometer (NanoDrop Technologies, Wilmington, Del.).

D. Genotype Analysis

Single nucleotide polymorphism genotyping was performed on an Applied Biosystems 7500 Real-Time Polymerase Chain Reaction System (Applied Biosystems, Foster City, Calif.). Assay reagents for each SNP were obtained from Applied Biosystems (FTO rs9939609, assay C_{—}30090620_{—}10; INSIG2 rs7566605, assay C_{—}29404113_{—}20). DNA was genotyped according to the manufacturer's protocol. Briefly, the components for each genotyping reaction were as follows: 10 ng of DNA, 5 μL of TaqMan Genotyping Master Mix (Applied Biosystems), 0.25 μL of assay mix (40×), and water up to a total volume of 10 μL. The thermocycler conditions were as follows: 50° C. for 2 minutes, 95° C. for 10 minutes, and 40 cycles at 95° C. for 15 seconds and at 60° C. for 60 seconds. The reaction was then analyzed using Applied Biosystems Sequence Detection Software.

E. Statistical Analysis

Deviation from Hardy-Weinberg equilibrium was tested with the HelixTree software package (Golden Helix, Bozeman, Mont.). The HelixTree application was used to determine differences in genotype and allele frequencies to examine the association of SNPs with BMI and laboratory results. Multiple testing corrections were performed using simulations and the Bonferroni method. Significant association was considered likely for a Bonferroni-corrected P<0.05.

### Results

A. Patient Characteristics

The mean age of the patient cohort was 45.9 years, with a mean BMI of 51.2 (Table 1). More than 97% of the patients had white European ancestry, representative of the geographic area, and 81% were women. Mean lipid measurements were as follows: triglyceride level, 177.6 mg/dL (2.01 mmol/L); total cholesterol, 188.8 mg/dL (4.89 mmol/L); high-density lipoprotein cholesterol, 48.1 mg/dL (1.25 mmol/L); total cholesterol to HDL cholesterol ratio, 4.1; and calculated low-density lipoprotein, 106.2 mg/dL (2.75 mmol/L). The distribution of BMI measurements is shown in FIG. 1. Almost 4% of the population had BMIs higher than 70.

TABLE 1
Characteristics of Patients Undergoing Roux-en-Y Gastric Bypass

| Characteristic | Value | |
|---|---|---|
| Body mass index^{a,b} | | |
| Mean (SD) | 51.2 | (8.5) |
| Median (range) | 49.5 | (40-88.4) |
| Triglycerides, mg/dL^{c} | | |
| Mean (SD) | 177.6 | (105.3) |
| Median (range) | 153 | (36-908) |
| Total cholesterol, mg/dL^{c} | | |
| Mean (SD) | 188.8 | (38.7) |
| Median (range) | 184 | (70-309) |
| HDL cholesterol, mg/dL^{c} | | |
| Mean (SD) | 48.1 | (11.3) |
| Median (range) | 47 | (22-103) |
| Total cholesterol to HDL cholesterol ratio^{c} | | |
| Mean (SD) | 4.1 | (1.1) |
| Median (range) | 4 | (1.5-7.8) |
| LDL cholesterol, mg/dL^{d} | | |
| Mean (SD) | 106.2 | (34) |
| Median | 102 | (5-226) |
| Age, y^{b} | | |
| Mean (SD) | 45.9 | (11.2) |
| Median (range) | 46.6 | (18.6-72.2) |

Abbreviations: HDL, high-density lipoprotein; LDL, low-density lipoprotein.
SI conversion factors: To convert HDL, LDL, and total cholesterol to millimoles per liter, multiply by 0.0259; triglycerides to millimoles per liter, multiply by 0.0113.
^{a}Calculated as weight in kilograms divided by height in meters squared.
^{b}N = 707.
^{c}n = 679.
^{d}n = 661.

B. Genotypes

A total of 707 DNA samples were genotyped for the FTO (rs9939609) and INSIG2 (rs7566605) SNPs (Table 2) Genotyping consisted of analyzing the DNA from each patient to determine whether he or she carried the A and/or T sequences in FTO and the G and/or C sequences near INSIG2. The FTO A SNP and the INSIG2 C SNP are considered the obesity SNPs. The frequencies of the INSIG2 and FTO SNPs in this population are presented in Table 2 and concur with previous studies.^{18,49}

TABLE 2
Frequencies of FTO and INSIG2 Alleles
in Morbidly Obese Patients

| Allele | No. of Alleles | Allele Frequency |
|---|---|---|
| FTO | | |
| A | 638 | 0.45 |
| T | 776 | 0.55 |
| INSIG2 | | |
| C | 495 | 0.35 |
| G | 919 | 0.65 |

Abbreviations: FTO, fat mass and obesity associated gene; INSIG2, insulin induced gene 2.

To determine whether the population was genetically skewed through inbreeding or strong founder effects, a statistical test for Hardy-Weinberg equilibrium was performed. Both SNPs were found to be well within Hardy-Weinberg equilibrium (FTO, P>0.44; INSIG2, P>0.29). Our frequency of SNP sequences is thus consistent with an out-bred, mixed, white European population.

The diploid SNP sequences, or genotypes (ie, AA, AT, and TT for FTO and CC, GC, and GG for INSIG2), of each patient for each gene were also analyzed (Table 3). The homozygous genotype AA in FTO was present in approximately 21% of the population and the homozygous genotype CC in INSIG2 was present in approximately 13%, consistent with previous studies.^{18,49} These 2 homozygous genotypes are considered the high-obesity risk genotypes. The heterozygous AT and GC genotypes were found in 48% and 44% of the study population, respectively. The homozygous low-obesity risk genotype for FTO (TT) was found in 31% of the population and the low-obesity risk genotype (GG) for INSIG2 was present in 43%.

TABLE 3
Frequencies of FTO and INSIG2 Genotypes
in Morbidly Obese Patients

| Genotype | No. of Genotypes | Genotype Frequency |
|---|---|---|
| FTO | | |
| AA | 149 | 0.21 |
| AT | 340 | 0.48 |
| TT | 218 | 0.31 |
| INSIG2 | | |
| CC | 93 | 0.13 |
| CG | 309 | 0.44 |
| GG | 305 | 0.43 |

Abbreviations: FTO, fat mass and obesity associated gene; INSIG2, insulin induced gene 2.

C. Association of BMI with SNPs

The relationship of BMI with the INSIG2 and FTO obesity SNP genotypes was analyzed using the HelixTree Genetics Analysis Software (Golden Helix). With this program, data are analyzed by minimizing the sum of squared deviations of each group mean from the remainder of the observations. An F test was used to generate an unadjusted P value; an adjusted P value was calculated by curve-fitting thousands of simulations; and a Bonferroni correction for multiple comparisons of the adjusted P value was also calculated. A conservative threshold of <0.05 was used for this Bonferroni-corrected P value.

The initial analysis was performed using BMI and the individual FTO and INSIG2 SNP genotypes. Although the mean BMIs increased by approximately 2 kg/m² in FTO and INSIG2 obesity genotypes (Table 4), they were not statistically different (Table 5). With a less stringent statistical threshold (not Bonferroni corrected), the BMIs of the 3 FTO genotypes were found to be significantly different (P=0.03). No significant association was found between the FTO or INSIG2 genotypes and any of the lipid parameters (P>0.10).

TABLE 4
Mean BMIs by FTO and INSIG2 Genotypes in Patients
Undergoing Roux-en-Y Gastric Bypass

| | FTO | | INSIG2 | |
|---|---|---|---|---|
| | Geno- | BMI, | Geno- | BMI, |
| Genotype Class | type | Mean (SD) | type | Mean (SD) |
| Homozygous for | TT | 50.4 (7.7) | GG | 50.7 (8.2) |
| normal weight | | | | |
| Heterozygous | AT | 51.1 (8.8) | GC | 51.3 (8.4) |
| Homozygous for obesity | AA | 52.5 (8.7) | CC | 52.5 (9.3) |

Abbreviations: BMI, body mass index (calculated as weight in kilograms divided by height in meters squared); FTO, fat mass and obesity associated gene; INSIG2, insulin induced gene 2.

TABLE 5
Significance of Association of Body Mass Index With FTO,
INSIG2, and Combined Genotypes in Morbidly Obese Patients

| | P Value | | |
|---|---|---|---|
| | | | Bonferroni |
| Genotype | Unadjusted | Adjusted | Corrected |
| FTO | .026 | .026 | .051 |
| INSIG2 | .106 | .274 | .824 |
| FTO and INSIG2 | <.001 | .003 | .010 |

Abbreviations: FTO, fat mass and obesity associated gene; INSIG2, insulin induced gene 2.

When both the FTO and the INSIG2 genotypes of each patient were considered together as a compound genotype (ie, AA/CC, AA/GC, AA/GG, AT/CC, AT/GC, AT/GG, AA/CC, AA/GC, and AA/GG), patients who were double homozygotes for the obesity-risk alleles (AA/CC) were found to have significantly higher BMIs (P<0.01, Bonferroni corrected). Those who were FTO homozygous and INSIG2 heterozygous (AA/GC) or FTO heterozygous and INSIG2 homozygous (AT/CC) for obesity also had significantly higher BMIs. No significant association was found between the compound FTO/INSIG2 genotypes and any of the lipid parameters (P>0.10).

An interesting pattern in mean BMI was found in the compound groups (Table 6). The mean BMI was about 4 kg/m² higher in the group homozygous for the obesity-risk genotypes (AA/CC) and was about 3 kg/m² higher in the homozygous/heterozygous (AA/GC) and the heterozygous/homozygous (AT/CC) groups compared with the other compound genotype groups. This is consistent with the contribution of an approximately 1 kg/m² increase in BMI for each copy of the FTO A and INSIG2 C obesity sequences in these groups. The association of at least 2 copies of 1 obesity SNP and at least 1 copy of the other with increased BMI also suggests some degree of interaction between FTO and INSIG2. However, biological factors appear to influence the observed data, because the group homozygous for normal weight/obesity (TT/CC, respectively) was approximately 2 kg/m² lower than the group homozygous for obesity/normal weight (AA/GG, respectively) and about 6 kg/m² lower than the group homozygous for obesity (AA/CC).

TABLE 6
Mean BMIs of FTO/INSIG2 Compound Groups

| | FTO/INSIG2 | | |
|---|---|---|---|
| Genotype Class | Genotype | BMI (SD) | |
| Normal weight/obesity | TT/CC | 48.6 | (5.6) |
| Normal weight/normal weight | TT/GG | 50.6 | (7.7) |
| Heterozygous/normal weight | TA/GG | 50.6 | (8.4) |
| Normal weight/heterozygous | TT/GC | 50.7 | (8.2) |
| Heterozygous/heterozygous | TA/GC | 50.8 | (8.6) |
| Obesity/normal weight | AA/GG | 50.9 | (8.4) |
| Obesity/Heterozygous | AA/GC | 53.0 | (8.3) |
| Heterozygous/Obesity | AT/CC | 53.5 | (10.0) |
| Obesity/obesity | AA/CC | 54.4 | (10.3) |

Abbreviations: BMI, body mass index (calculated as weight in kilograms divided by height in meters squared); FTO, fat mass and obesity associated gene; INSIG2, insulin induced gene 2.

### Discussion

Obesity is a multifactorial condition, with substantial evidence supporting a strong genetic component.⁴⁷ Such genetic factors may influence therapies, including bariatric surgery; thus, their identification may be important in guiding treatment. Mutations in several genes have been found to be responsible for rare familial monogenic forms of obesity, and a large number of genes have been analyzed in common sporadic multigenic obesity.¹³ However, many studies of genes in common obesity have not been replicated across different populations.²¹

The 2 obesity gene variants studied here, rs9939609 (FTO) and rs7566605 (INSIG2), have previously been replicated in multiple, but not all, studies. For example, the INSIG2 variant was first replicated in 4 separate cohorts composed of individuals with Western European ancestry, African American individuals, and children,¹⁴ but later, it was found to have both negative^{49,50-54} and positive¹⁵ associations in genetic analyses of several thousand individuals. There have been fewer studies of the FTO variant, though the data have been largely supportive of its association with BMI.^{19,20-55} These inconsistent results may be because the effect each SNP variant has on BMI is relatively small and could be influenced by slight differences in population characteristics and gene-gene and gene-environment interactions. Our results support the possibility that gene-gene interactions are important, because the strongest association with BMI occurred when both genes were analyzed together. No previous studies have examined the combined effects of the FTO and INSIG2 SNPs in obesity.

Despite the large number of participants analyzed in studies examining the association of BMI with either the INSIG2 or FTO SNPs, the populations have largely comprised individuals with normal weight, overweight, and class I obesity (BMI>30 and <35). The range of BMIs in most of the previous studies was less than 20 (˜20-40) compared with a range of more than 45 in our population (˜40-88). However, the studies showing an association between obesity and the INSIG2 SNP have tended to have populations with higher BMIs.⁴⁹ Similarly, an SNP in FTO, near but different than the SNP analyzed here, has been associated with BMI in a population of morbidly obese adults.¹⁸

The homozygous/homozygous, homozygous/heterozygous, and heterozygous/homozygous compound FTO/INSIG2 SNP genotypes that were associated with higher BMIs were present in less than 20% of the cohort, indicating that a potentially large number of other genes that influence BMI in the morbidly obese are not yet identified. Candidate genes include those previously associated with obesity in rare monogenic forms of the condition and those involved in obesity based on cell biological or animal model studies.⁵⁶ However, few new sequence variants were found in previously identified obesity candidate genes using a morbidly obese population similar to that studied here.⁴⁸ These results indicate that sporadic morbid obesity is likely to be influenced by other, unidentified genes, not candidate genes selected by biological inference.^{36,57,58}

Other factors that may also affect the influence of genetic variants on morbid obesity include sex and race, which were not addressed in the predominantly white female population studied here. The prevalence of morbid obesity is higher in women than in men and in individuals of African ancestry compared with white or Hispanic individuals,⁶⁰ with the lowest prevalence in Asian persons.⁸ Two of the studies that found no association of B1MI with the INSIG2 genotype were conducted in patients with primarily African ancestry.^{15,5} Data from the International HapMap Project indicate that the high-obesity risk INSIG2 CC genotype was present at a higher frequency in the Japanese and Han Chinese populations analyzed than in the European and African groups.⁶¹ In contrast, the high-obesity risk FTO AA genotype was present at a lower frequency in the 2 Asian populations compared with the European and African groups. These data suggest that the effects of the 2 obesity SNPs may not be similar in all racial groups and that further studies will be needed to address other populations.

A limitation of association studies is that potential causative mechanisms cannot be identified, thus the potential pathophysiological role(s) of the FTO and INSIG2 genes in morbid obesity are not known; INSIG2 codes for an endoplasmic reticulum protein that regulates the movement of sterol regulatory element, binding proteins to the Golgi apparatus and regulating the synthesis of fatty acid and cholesterol.^{16,62} Overexpression of Insig2 in the liver of rats reduced plasma triglyceride levels.¹⁷ Despite this clear involvement in lipid metabolism, no association between common lipid parameters and the INSIG2 (or FTO) SNPs was found here. However, medication use was not accounted for in the analysis, thus the effects of lipid-lowering agents may have affected the phenotypes of those genetically predisposed to dyslipidemia. The function of the protein product of FTO has not yet been elucidated. Mice with the Fto syntenic fused toes mutation manifest developmental defects.⁶³⁻⁶⁴

How the SNPs in INSIG2 and PTO alter the function of their respective RNAs and/or proteins, increasing the risk for higher BMI in the morbidly obese, is not yet known. The INSIG2 SNP is located about 10 000 base pairs upstream from the coding region, so it is likely involved in regulating the level of RNA and therefore the amount of protein produced. The FTO SNP is located in the first intron of the gene and also presumably affects levels of its RNA and protein. Future studies will be required to determine the molecular mechanism through which the specific DNA sequences, ie, A and T for FTO and G and C for INSIG2, affect the genes' functions. Our results indicate that the 2 genes may interact, suggesting that the physiological pathways in which each is involved may be linked in some way.

Surgical treatment for morbidly obese patients results in greater weight loss than medical treatment does.¹ Bariatric surgery has also been associated with increased life expectancy compared with the risk of surgical mortality and potential length of effectiveness.⁶⁵ Recent data on the long-term effectiveness of bariatric surgery on BMI⁴⁶ suggest that, for most patients, BMI will be maintained substantially below preoperative levels, though some patients regain weight and relapse toward morbid obesity. Genetic susceptibility alleles that overcome the results of the Roux-en-Y gastric bypass surgery, have been investigated for several candidate genes in laparoscopic adjustable-band therapy and laparoscopic mini-gastric bypass.^{37,66} The identification of such susceptibility genes is therefore important in identifying patients at high risk for postoperative weight gain. These studies may also represent some of the first specific examples of “surgicogenomics,” paralleling the well-developed field of pharmacogenomics.⁶⁷

### Example 2

### Association of FTO, INSIG2, MC4R, and PCSK1 SNPs with BMI

It was hypothesized that SNPs that confer susceptibility to obesity are also related to resistance to weight loss therapies. Genetic factors play an important role in the regulation of body weight as well as in the development of obesity¹². In addition to a Mendelian variants that have been associated with obesity¹³, common variants in several genes have also been found through genome-wide association studies (GWAS). One of the first SNPs related to BMI found through GWAS resides near the insulin signaling protein type 2 (INSIG2) gene^{14,15}, involved in lipid and cholesterol metabolism¹⁶ and linked to obesity in rodents¹⁷. Another obesity SNP resides within the FTO (fat mass and obesity associated) gene (rs9939609)^{18,19}, further validated through meta analysis and other studies^{20,21}. Another large-scale meta-analysis of GWAS data identified a SNP nearby the coding sequence of MC4R²². Rare coding mutations in the MC4R gene are a leading cause of monogenic obesity in humans^{23,24}. Mutations in PCSK1 also cause monogenic obesity, and a SNP producing a nonsynonymous variant was associated with obesity in adults and children of European ancestry.

It was hypothesized that common genetic variants that predispose patients to obesity would also be related to less weight loss following gastric bypass surgery. The association of genotypes of four obesity SNPs with weight loss from dietary regimens and bariatric surgery was analyzed, and with behavioral and metabolic data, in a cohort of severely obese patients.

### Methods

A. Study Population

All patients who were enrolled in the Bariatric Surgery Program of the Geisinger Center for Nutrition and Weight Management were recruited into a clinical research program in obesity²⁵. A comprehensive medical history and physical examination was performed during the initial visit. Patients undergo a pre-operative assessment and preparation period during which a comprehensive set of clinical and laboratory measures were obtained along with blood samples for serum and DNA isolation. All patients who were enrolled in the bariatric surgery program were placed on a 6-8 month pre-operative assessment and preparation period, that is designed to produce a weight loss of at least 3%, with a target of 10%, without the use of weight loss medication. Patients were placed on a prudent diet with a 500-700 kcal deficit with support that included individual and group sessions with monthly meetings and sessions of nutrition and physical activity education and social support for four months. Patients who failed to lose at least 3 percent of their body weight after 4 months on the hypocaloric diet were prescribed a liquid diet. The liquid diet consisted of a high protein shake instead of eating meals, and a very low calorie intake of ˜1000 calories a day, with a goal of rapid weight loss of about 3-4 pounds a week for the 2 months prior to surgery. After completion of the pre-operative program, all patients underwent a Roux-en-Y gastric bypass procedure. All patients were followed at 1-3 month intervals up to three years after surgery. The Institutional Review Board of the Geisinger Clinic approved the research protocol and all participants provided written informed consent. Clinical data were extracted from the EpicCare EHR and read into SAS/STAT software (SAS Institute Inc., Cary, N.C.) as described elsewhere²⁶.

B. Genotyping

DNA was extracted from 0.35 ml of EDTA anti-coagulated whole blood using the Qiagen MagAttract DNA Blood Midi M48 Kit and Qiagen BioRobot M48 Workstation (Qiagen, Valencia, Calif.) according the manufacturer's directions. The final elution volume was 200 ul. For a small number of patients, blood was not available so DNA was extracted from fixed liver tissue. Liver was first treated with proteinase K (lug/ul) in 350 ul Qiagen Tissue Lysis Buffer and incubated at 55° C. overnight. Following digestion, samples were loaded to Qiagen BioRobot M48 Workstation and extracted for DNA as described above for blood samples. Quantification of DNA extracted was performed using a NanoDrop ND-1000 spectrophotometer (NanoDrop Technologies, Wilmington, Del.).

Genotype analysis: Single nucleotide polymorphism (SNP) genotyping was performed on an Applied Biosystems 7500 real-time PCR System (Applied Biosystems, Foster City, Calif.). Assay reagents for each SNP were obtained from Applied Biosystems (INSIG2, rs7566605, Assay ID: C_{—}29404113_{—}20; FTO, rs9939609, Assay ID: C_{—}30090620_{—}10; MC4R, rsl7782313, C_{—}32667060_{—}10; PCSK1, rs6235, C_{—}2841942_{—}10). DNA was genotyped according to the manufacturer's protocol. Briefly, the reaction components for each genotyping reaction were as follows: 10 ng of DNA, 5 μL of TaqMan Genotyping Master Mix (Applied Biosystems, Foster City, Calif.), 0.25 μL of assay mix (40×), and water up to a total volume of 10 μL. The thermocycler conditions were as follows: 50° C. for 2 min, 95° C. for 10 min, and 40 cycles of 95° C. for 15 sec and 60° C. for 60 sec. The reaction was then analyzed by Applied Biosystems Sequence Detection Software.

C. Eating Behavior

Each subject completed a questionnaire based on the Diagnostic and Statistical Manual of Mental Disorders, 4th edition, diagnostic criteria for binge-eating disorder²⁸ using the fully validated eating disorder questionnaire of Spitzer et al.³⁰ (German translation³⁴). To validate the questionnaire data, a certified dietitian and a psychologist conducted independent, semistructured interviews with each subject. Finally, the physician specializing in obesity conducted a structured interview using this questionnaire. Team members were unaware of the subjects' behavioral diagnosis and genotype. Unanimity among all three professionals characterizing each subject's eating behavior was required for a diagnosis of binge eating. Diagnostic criteria for binge eating included at least twice-weekly binge eating over a minimum of six months. A binge was defined as rapid consumption of an unusually large amount of food in the absence of hunger, causing the subject to feel embarrassed, depressed, or guilty and out of control. There was no purging behavior. Subjects who did not fulfill all criteria for binge-eating disorder, determined unanimously by the team, were described as “non-bingers.”

D. Resting Energy Expenditure

Resting energy expenditure and diet-induced thermogenesis (defined as the excess energy expended after a standard meal and expressed as a percentage of resting energy expenditure) were determined from continuous indirect calorimetry for three hours after the meal.

E. Statistical Analysis

A two-tailed significance level of 0.05 was used. SAS version 9.1 was used for all data manipulations and statistical analysis. In initial analyses, graphical displays and frequency distributions were constructed to describe the study population. Multiple findings were evaluated for coherence and sense according to scientific plausibility, rather than by focusing on individual p-values.

For the bivariate analysis of type of dietary weight loss regimen with number of homozygous obesity genotypes and with number obesity alleles, a Wilcoxon Rank Sum test was used. In multivariate analysis, logistic regression models was used to determine if dietary weight loss regimen is predicted by genotype pattern (i.e. number of homozygous obesity genotypes and/or number of obesity alleles) after controlling for other patient characteristics (i.e. gender, age, baseline BMI, etc.). These approaches were selected because they enable the correlation of a dichotomous variable (i.e. dietary weight loss regimen) with an ordinal variable (i.e. genotype pattern). This analysis technique was used to compare the two dietary weight loss regimens of the pre-operative period (i.e. prudent hypocaloric diet and liquid diet). For prudent hypocaloric diet weight loss analysis, patients that lost at least 3% of body weight in the hypocaloric weight loss program (expected to be N=1000) were compared versus patients that did not and are prescribed the liquid diet intervention weight loss program (expected to be N=1000). For analysis of the ˜1000 patients who are patients that are resistant to the prudent hypocaloric diet, the patients that are resistant to the liquid diet (expected N=600) were compared versus patients that respond to the liquid diet (expected N=400). As a secondary analysis for this aim, the mean pre-surgery weight loss was correlated with genotype data using linear regression.

For the outcome variable of percent excess body weight loss post-surgery, two definitions were used based upon common clinical use. One outcome was due to measure excess body weight loss as a single point at either 12 or 24 months post-surgery. The mean percent excess weight loss at 12 and 24 months was be correlated with genotype data (i.e. number of homozygous obesity genotypes and/or number of obesity alleles) using a linear regression model. Overall F-tests, tests of trend, and pairwise comparisons (after using a Bonferroni correction), were considered in the analysis. The other outcome will be time until goal weight loss of at either 50%, 60% and/or 70% excess body weight. The time until goal weight loss (i.e. loss of >50% of excess body weight post-surgery), was correlated with genotype data using Kaplan-Meier survival curves and Cox regression.

### Results

A. Patient characteristics

A total of 1062 caucasian patients with a mean age of 46.5 years and a mean BMI of 50.1 mg/kg2 (Table 7) who underwent both a pre-operative weight loss regimen and gastric bypass surgery were genotyped for the INSIG2, FTO, MC4R, and PCSK1 obesity SNPs. Data is thus available on dietary and surgical weight loss outcomes on the same patient. Patients were categorized as homozygous obese if they were homozygous for the obesity high risk allele for each SNP, heterozygous obese if they were carriers of the obesity risk allele, and homozygous non-obese if they were homozygous for the low risk allele. The frequencies of the minor alleles for each of the 4 obesity SNPs reported for control populations^{27,28} were in good agreement with the results here. All 4 SNPs were found to be well within Hardy-Weinberg equilibrium ((INSIG2: p=0.66, FTO: p=0.54, MC4R: p=0.73, PCSK1: p=0.73). The frequency of the SNP alleles is thus consistent with an outbred mixed Caucasian/European population. Patients were also categorized by how many homozygous SNP genotypes they had and by the total number of obesity risk alleles they possessed whether in the homozygous or heterozygous configuration. Less than one percent of the population had 3 or more homozygous genotypes, while less than three percent carried 6 or more risk alleles.

TABLE 7
Demographic, anthropometric, and genotype characteristics
of the study population (N = 1062).

| | | | |
|---|---|---|---|
| Age | Mean (SD) | 46.5 | (11.0) |
| | Median [Range] | 46 | [18, 72] |
| Gender | Female (%) | 854 | (80%) |
| | Male (%) | 208 | (20%) |
| Ethnicity/Race | White (Non-Hispanic) | 1109 | (100%) |
| Height, inches | Mean (SD) | 65.5 | (3.5) |
| Weight, lbs | Baseline, mean (SD) | 307 | (62) |
| | Surgery, mean (SD) | 293 | (60) |
| BMI, kg/m² | Baseline, mean (SD) | 50.2 | (8.7) |
| | Surgery, mean (SD) | 47.9 | (8.3) |
| Excess weight, lbs | Baseline, mean (SD) | 154 | (56) |
| | Surgery, mean (SD) | 140 | (53) |
| INSIG2* | Homozygous obese (%) | 136 | (13%) |
| | Heterozygous (%) | 450 | (42%) |
| | Homozygous normal (%) | 476 | (45%) |
| FTO* | Homozygous obese (%) | 231 | (22%) |
| | Heterozygous (%) | 518 | (49%) |
| | Homozygous normal (%) | 313 | (29%) |
| MC4R* | Homozygous obese (%) | 80 | (8%) |
| | Heterozygous (%) | 401 | (38%) |
| | Homozygous normal (%) | 581 | (55%) |
| PCSK1* | Homozygous obese (%) | 87 | (8%) |
| | Heterozygous (%) | 411 | (39%) |
| | Homozygous normal (%) | 564 | (53%) |
| # of homozygous | 0 (%) | 623 | (59%) |
| obese genotypes | 1 (%) | 349 | (33%) |
| | 2 (%) | 85 | (8%) |
| | 3 (%) | 5 | (<1%) |
| # of obesity alleles | 0 (%) | 45 | (4%) |
| | 1 (%) | 158 | (15%) |
| | 2 (%) | 289 | (27%) |
| | 3 (%) | 293 | (28%) |
| | 4 (%) | 179 | (17%) |
| | 5 (%) | 73 | (7%) |
| | 6 (%) | 23 | (2%) |
| | 7 (%) | 2 | (<1%) |

*Test for deviation from Hardy-Weinberg equilibrium was not significant (p > 0.05)

B. Association with Dietary Weight Loss

The association of the FTO, INSIG2, MC4R, and obesity SNPs with weight loss outcomes from dietary interventions was determined.²⁵ Patients were stratified by genotype based upon the number of FTO, INSIG2, MC4R, and/or PCSK1 obesity SNPs they carried, irrespective of dosage, thus patients had from 0-8 alleles (Table 8). The data was also analyzed with respect to the number of homozygous SNPs they carried, thus patients had from 0-4 homozygous genotypes. No relationship was present between either number of obesity alleles or homozygous obesity genotypes and the average amount of weight lost from dietary interventions, consistent with other studies²⁹. The population was then further analyzed based upon whether the patients were placed on a liquid diet following failure to lose weight on the initial program of a hypocaloric diet.

TABLE 8
Mean percent of excess weight loss prior
to surgery by genotype (N = 1062).

| | | Mean Weight Loss | | |
|---|---|---|---|---|
| | N | (SD) | | p-value |
| INSIG2 | | | | 0.85¹ |
| Homozygous obese | 136 | 8.8% | (10.4) | |
| Heterozygous | 450 | 9.4% | (9.6) | |
| Homozygous normal | 476 | 9.1% | (9.5 | |
| FTO | | | | 0.39¹ |
| Homozygous obese | 231 | 9.1% | (9.0) | |
| Heterozygous | 518 | 8.9% | (9.9) | |
| Homozygous normal | 313 | 9.8% | (9.8) | |
| MC4R | | | | 0.85¹ |
| Homozygous obese | 80 | 9.6% | (8.9) | |
| Heterozygous | 401 | 9.3% | (9.7) | |
| Homozygous normal | 581 | 9.1% | (9.8) | |
| PCSK1 | | | | 0.87¹ |
| Homozygous obese | 87 | 8.8% | (11.7) | |
| Heterozygous | 411 | 9.4% | (9.1) | |
| Homozygous normal | 564 | 9.1% | (9.7) | |
| # of homozygous obese genotypes | | | | 0.67² |
| 0 | 623 | 9.5% | (9.7) | |
| 1 | 349 | 8.5% | (9.3) | |
| 2+ | 90 | 9.9% | (10.6) | |
| # of obesity alleles | | | | 0.48² |
| 0 | 45 | 9.3% | (10.9) | |
| 1 | 158 | 9.8% | (9.4) | |
| 2 | 289 | 9.3% | (9.7) | |
| 3 | 293 | 8.6% | (9.3) | |
| 4 | 179 | 9.5% | (10.1) | |
| 5 | 73 | 8.2% | (9.9) | |
| 6+ | 25 | 11.3% | (9.6) | |
| # of obesity alleles (version #2) | | | | 0.80² |
| 0 | 45 | 9.3% | (10.9) | |
| 1-2 | 447 | 9.4% | (9.6) | |
| 3-4 | 472 | 9.0% | (9.6) | |
| 5+ | 98 | 9.0% | (9.9) | |

¹One-way ANOVA;
²Test for linear trend

C. Association with Post-Operative Weight Loss

A similar analysis was conducted using weight loss data at 12 months following Roux-en-Y Gastric Bypass surgery calculated as excess body weight, an estimate of fat mass used for assessing weight loss that is based upon based upon an idealized BMI of 25 kg/m². Patients were again stratified by genotype based upon the number of FTO, INSIG2, MC4R, and/or PCSK1 obesity SNPs they carried (Table 9). In contrast to diet-induced weight loss, a statistically significant trend was present between a decreasing amount of excess body weight lost with increasing number of obesity SNP alleles (p=; F-test statistic=). The data was also analyzed with respect to the number of homozygous SNPs patients carried (Table 9), which also exhibited a statistically significant trend (p=; F-test statistic=).

TABLE 9
Mean percent of excess body weight lost at 12 (N = 885) and 24 (N = 516) months post surgery.

| | 12-month follow-up | | | 24-month follow-up | | |
|---|---|---|---|---|---|---|
| | N | Mean (SD) | p-value | N | Mean (SD) | p-value |
| INSIG2 | | | 0.21¹ | | | 0.11¹ |
| Homozygous obese | 110 | 65.4% (21.8) | | 67 | 63.1% (26.9) | |
| Heterozygous | 385 | 68.8% (22.7) | | 226 | 69.9% (22.1) | |
| Homozygous normal | 397 | 69.0% (23.1) | | 223 | 69.9% (25.9) | |
| FTO | | | 0.33¹ | | | 0.20¹ |
| Homozygous obese | 202 | 66.7% (23.0) | | 116 | 65.6% (22.3) | |
| Heterozygous | 432 | 68.6% (22.2) | | 246 | 70.5% (24.5) | |
| Homozygous normal | 258 | 69.8% (23.5) | | 154 | 69.2% (26.0) | |
| MC4R | | | 0.99¹ | | | 0.78¹ |
| Homozygous obese | 71 | 68.7% (22.6) | | 39 | 70.0% (29.6) | |
| Heterozygous | 332 | 68.6% (22.7) | | 187 | 68.0% (23.1) | |
| Homozygous normal | 489 | 68.4% (22.9) | | 290 | 69.5% (24.7) | |
| PCSK1 | | | 0.45¹ | | | 0.73¹ |
| Homozygous obese | 74 | 65.6% (17.2) | | 38 | 66.2% (19.4) | |
| Heterozygous | 344 | 68.2% (23.2) | | 200 | 69.7% (24.7) | |
| Homozygous normal | 474 | 69.1% (23.3) | | 278 | 68.9% (25.0) | |
| # of homozygous obese genotypes | | | 0.065² | | | 0.043² |
| 0 | 519 | 69.6% (23.3) | | 306 | 70.9% (24.6) | |
| 1 | 294 | 67.5% (22.3) | | 163 | 67.3% (24.0) | |
| 2+ | 79 | 64.5% (20.9) | | 47 | 63.1% (25.2) | |
| # of obesity alleles | | | 0.12² | | | 0.031² |
| 0 | 33 | 74.5% (21.1) | | 22 | 79.7% (32.4) | |
| 1 | 135 | 68.2% (24.5) | | 83 | 64.9% (24.1) | |
| 2 | 242 | 69.8% (23.5) | | 139 | 72.1% (25.3) | |
| 3 | 248 | 68.0% (22.3) | | 139 | 69.3% (21.5) | |
| 4 | 152 | 68.0% (22.3) | | 82 | 68.2% (26.0) | |
| 5 | 58 | 66.7% (20.2) | | 35 | 63.1% (22.1) | |
| 6+ | 24 | 60.1% (20.3) | | 16 | 63.6% (26.4) | |
| # of obesity alleles (version #2) | | | 0.032² | | | 0.0084² |
| 0 | 33 | 74.5% (21.1) | | 22 | 79.7% (32.4) | |
| 1-2 | 377 | 69.2% (23.9) | | 222 | 69.4% (25.0) | |
| 3-4 | 400 | 68.0% (22.3) | | 221 | 68.9% (23.2) | |
| 5+ | 82 | 64.8% (20.4) | | 51 | 63.3% (23.3) | |

^{l}One-way ANOVA;
²Linear regression
**12-month follow-up was defined as the weight occurring closest to 12 months from surgery but between 9 and 18 months post surgery.
**24-month follow-up was defined as the weight occurring closest to 24 months from surgery but between 19 and 30 months post

D. Interaction of BMI and Genotype on Weight Loss Dynamics

The obesity SNPs analyzed are related to increased BMI, thus it was determined whether BMI interacted with genotype to affect weight loss. The population was divided approximately in half into a group with BMI<50 and a group with BMI>50, the threshold for “super obesity”. The impact of genotype on weight loss during the 6 month pre-operative program and on weight loss up to 30 months post-operatively was modeled for these two groups Neither BMI nor genotype had an effect upon diet-induced weight loss. However, a dramatic effect was present for post-operative weight loss. Patients with 2+ homozygous genotypes (FIG. 2) or 5+ obesity alleles (FIG. 3) experienced substantial weight gain following an initial steep post-operative weight loss. These groups lost approximately 50% EBWL at 30 months after surgery, with a slope indicating the potential further weight gain. In contrast, obesity SNPs were unrelated to post-operative weight loss in patients with BMI>50. One hypothesis is that other genes may blunt the effects of the common obesity alleles at the upper extremes of BMI.

E. Association with Behavior and Metabolism

Data relevant to eating behavior and metabolism were available on a subset of patients that were used to address potential mechanisms for how obesity SNPs may impact weight loss. The four obesity SNPs were analyzed in relation to binge eating behavior as diagnosed using the Questionnaire on Eating and Weight Patterns-Revised (QEWP-R)³⁰. A total of 120 (18%) patients were classified as manifesting binge-eating behavior (Table 10). No association was found with additive, recessive, dominant, or allelic models for FTO, MC4R, or PCSK1. For INSIG2, however, an increased frequency of binge eating was found with both additive (not shown) and recessive models (Table 10) for the homozygous obesity genotype.

TABLE 10
Frequency of Obesity SNPs and QEWP-R Eating Behaviors

| | | | Eating behavior | | | | | | |
|---|---|---|---|---|---|---|---|---|---|
| | | | | | Episodic | | | | |
| | | | Normal | | overeating | | Binge eating | | |
| Gene (SNP) | Genotype | N | no. (%) | | no. (%) | | no. (%) | | P value* |
| INSIG2 | CC (Homozygous obesity) | 87 | 53 | (61%) | 9 | (10%) | 25 | (29%) | 0.013 |
| (rs7566605) | C/X (Heterozygous obesity or | 578 | 386 | (67%) | 97 | (17%) | 95 | (16%) | |
| | homozygous normal) | | | | | | | | |
| FTO | AA (Homozygous obesity) | 146 | 95 | (65%) | 27 | (18%) | 24 | (16%) | 0.59 |
| (rs9939609) | A/X (Heterozygous obesity or | 519 | 344 | (66%) | 79 | (15%) | 96 | (19%) | |
| | homozygous normal) | | | | | | | | |
| MC4R | CC (Homozygous obesity) | 57 | 34 | (60%) | 10 | (18%) | 13 | (23%) | 0.53 |
| (rs17782313) | C/X (Heterozygous obesity or | 608 | 405 | (67%) | 96 | (16%) | 107 | (18%) | |
| | homozygous normal) | | | | | | | | |
| PCSK1 | CC (Homozygous obese) | 56 | 33 | (59%) | 14 | (25%) | 9 | (16%) | 0.15 |
| (rs6235) | C/X (Heterozygous obesity or | 609 | 406 | (67%) | 92 | (15%) | 111 | (18%) | |
| | homozygous normal) | | | | | | | | |

*P value based upon Chi-square test.
All 4 SNPs were genotyped using Applied Biosystems Taqman SNP Genotyping assays and did not deviate from Hardy-Weinberg equilibrium (P > 0.05).

To determine whether genotype was associated with altered basal metabolism, resting energy expenditure (REE) was measured using indirect calorimetry in 536 patients. The results are shown in Table 11. The INSIG2 SNP may be associated with an obesity related eating behavior and not with a differences in basal metabolism, as suggested by Chung and Leibel.³¹

TABLE 11
Mean resting energy expenditure (REE) prior to surgery
by genotype (N = 847 with non-missing values).

| | N | Mean (SD) | p-value |
|---|---|---|---|
| INSIG-2 | | | 0.030¹ |
| Homozygous obese | 101 | 2518 (585) | |
| Heterozygous | 364 | 2528 (774) | |
| Homozygous normal | 382 | 2395 (688) | |
| FTO | | | 0.56¹ |
| Homozygous obese | 189 | 2512 (724) | |
| Heterozygous | 395 | 2465 (741) | |
| Homozygous normal | 263 | 2438 (677) | |
| MC4R | | | 0.20¹ |
| Homozygous obese | 60 | 2531 (735) | |
| Heterozygous | 318 | 2411 (689) | |
| Homozygous normal | 469 | 2496 (733) | |
| PSK1 | | | 0.42¹ |
| Homozygous obese | 74 | 2525 (806) | |
| Heterozygous | 332 | 2429 (703) | |
| Homozygous normal | 441 | 2486 (713) | |
| # of homozygous obese genotypes | | | 0.24² |
| 0 | 496 | 2431 (712) | |
| 1 | 281 | 2513 (743) | |
| 2+ | 70 | 2539 (642) | |
| # of obesity alleles | | | 0.25² |
| 0 | 36 | 2398 (522) | |
| 1 | 135 | 2474 (715) | |
| 2 | 226 | 2416 (735) | |
| 3 | 225 | 2472 (730) | |
| 4 | 149 | 2506 (740) | |
| 5 | 58 | 2555 (693) | |
| 6+ | 18 | 2508 (642) | |
| # of obesity alleles (version #2) | | | 0.27² |
| 0 | 36 | 2398 (522) | |
| 1-2 | 361 | 2438 (727) | |
| 3-4 | 374 | 2486 (733) | |
| 5+ | 76 | 2544 (678) | |

¹One-way ANOVA;
²Test for linear trend

### Discussion

This study sought to determine whether previously identified SNPs known to be associated with obesity were related to weight loss outcomes from a short-term dietary program and following bariatric surgery. An increasing number of obesity alleles of four obesity genes (INSIG2, FTO, MC4R, and PCSK1) were associated with decreased weight loss following bariatric surgery, with no association found with dietary weight loss. The effect of genotype is not present in patients with BMI>50. In addition, homozygosity for the INSIG2 obesity SNP was associated with binge eating behavior, with no relationship of genotype found with basal metabolic rate.

The results indicate that obesity SNPs that are associated with weight loss from bariatric are not associated with dietary/weight loss interventions. Previous studies have focused primarily on individual obesity SNPs and their relationship to dietary/lifestyle weight loss. Homozygosity for the INSIG2 obesity SNP was found to be associated with lower weight loss in a lifestyle intervention program in children³². During a 4-year follow-up, the FTO obesity SNP did not modify the of magnitude of weight reduction during a long-term lifestyle intervention in the Finnish Diabetes Prevention Study (DPS)³³. In 1,466 German subjects³⁴ with increased risk for type 2 diabetes, there was also no influence of the FTO polymorphism on changes in body weight or fat distribution during a lifestyle intervention. Children with certain MC4R mutations were able to lose weight in a lifestyle intervention program but had much greater difficulties to maintain weight loss³⁵. In aggregate these studies are consistent our results that obesity alleles are not associated With dietary/lifestyle weight loss.

Several previous studies have also examined candidate genes in relation to bariatric surgery. Weight loss at the 6-month follow-up after laparoscopic gastric banding was related to polymorphisms in interleukin 6 (IL6) and UCP2 genes in a study of 167 patients³⁶. UCP2 SNPs were also related to weight loss outcomes following gastric banding and gastric bypass in a study of 304 patients and 304 controls³⁷. Neither a UCP3 promotor nor a tumor necrosis factor (TNF) alpha polymorphism were related to weight loss outcomes 1 year after biliopancreatic diversion in studies of 40 morbidly obese patients³⁸. Similarly, SNPs in two g protein genes, GNB3 and GNAS1 were not related to weight loss following gastric banding in a study of 304 patients³⁹. These studies suffer from small sample size, as well as short length of follow-up. Our study supports a polygenic contribution to weight gain following bariatric surgery.

The effect of BMI on the association of the obesity SNPs with post-operative weight loss was unexpected. The common obesity variants were not implicated in weight loss in “super obese” patients. The patients at this extreme BMI may represent a group that is affected by other as yet unknown genetic factors that over-ride the contribution of the common variants. For example, rare loss of function mutations in the MC4R gene may be present in adult extremely obese patients′. Alternatively, the “super obese” may be influenced by some as yet unidentified environmental factors.

The mechanism by which obesity alleles affect BMI may also impact weight loss. An FTO obesity risk allele (rs8050136) was significantly associated with higher energy intake during dietary restriction, but not with resting energy expenditure⁴¹. In another study, an FTO obesity SNP was related to energy intake and preference for foods of high caloric density in 76 children, but was not associated with resting energy expenditure (REE),⁴². In a large Danish study, an interaction between the FTO rs9939609 genotype and physical activity was found⁴³. Binge eating was initially found to be a major phenotypic characteristic of subjects with a mutation in MC4R⁴⁴, although subsequent studies have not found such an association^{40,45}. Our data indicate that binge eating may be a factor because of the association in patients homozygous for the INSIG2 obesity SNP. Further studies will be required to delineate the mechanisms underlying the influence of obesity genes on weight loss following bariatric surgery.

In summary, the association between genetic variants in four genes related to obesity and weight loss from either dietary or surgical interventions was evaluated. An accumulating allele burden was associated with poorer outcomes following bariatric surgery in patients with BMI<50.
1. Maggard M A, Shugarman L R, Suttorp M et al. Meta-analysis: surgical treatment of obesity. Ann Intern Med 2005; 142:547-59.
2. Dixon J B, O'Brien P E, Playfair J et al. Adjustable gastric banding and conventional therapy for type 2 diabetes: a randomized controlled trial. Jama 2008; 299:316-23.
3. Cummings S, Apovian C M, Khaodhiar L. Obesity surgery: evidence for diabetes prevention/management. J Am Diet Assoc 2008; 108:S40-4.
4. Mark A L. Dietary therapy for obesity is a failure and pharmacotherapy is the future: a point of view. Clin Exp Pharmacal Physiol 2006; 33:857-62.
5. Elder K A, Wolfe B M, Bariatric surgery: a review of procedures and outcomes. Gastroenterology 2007; 132:2253-71.
6. Willer C J, Speliotes E K, Loos R J et al. Six new loci associated with body mass index highlight a neuronal influence on body weight regulation. Nat Genet 2008.
7. Ogden C L, Yanovski S Z, Carroll M D, Flegal K M. The epidemiology of obesity. Gastroenterology 2007; 132:2087-102.
8. McTigue K, Larson J C, Valoski A et al. Mortality and cardiac and vascular outcomes in extremely obese women. Jama 2006; 296:79-86.
9. Mirabelli D, Chiusolo M, Ferrante D, Balzola F, Merletti F, Petroni M L. Long-term mortality in a cohort of severely obese persons in Italy. Obesity (Silver Spring) 2008; 16:1920-5.
10. Pi-Sunyer F X. How effective are lifestyle changes in the prevention of type 2 diabetes mellitus? Nutr Rev 2007; 65:101-10.
11. Lanyon R I, Maxwell B M. Predictors of outcome after gastric bypass surgery. Obes Surg 2007; 17:321-8.
12. Bell C G, Walley A J, Froguel P. The genetics of human obesity. Nat Rev Genet 2005; 6:221-34.
13. Rankinen T, Zuberi A, Chagnon Y C et al. The human obesity gene map: the 2005 update. Obesity (Silver Spring) 2006; 14:529-644.
14. Herbert A, Gerry N P, McQueen M B et al. A common genetic variant is associated with adult and childhood obesity. Science 2006; 312:279-83.
15. Lyon H N, Emilsson V, Hinney A et al. The association of a SNP upstream of INSIG2 with body mass index is reproduced in several but not all cohorts. PLoS Genet 2007; 3:e61.
16. Yabe D, Brown M S, Goldstein J L. Insig-2, a second endoplasmic reticulum protein that binds SCAP and blocks export of sterol regulatory element-binding proteins. Proc Natl Acad Sci USA 2002; 99:12753-8.
17. Takaishi K, Duplomb L, Wang M Y, Li J, Unger R H. Hepatic insig-1 or -2 overexpression reduces lipogenesis in obese Zucker diabetic fatty rats and in fasted/refed normal rats. Proc Natl Acad Sci USA 2004; 101:7106-11.
18. Dina C, Meyre D, Gallina S et al. Variation in FTO contributes to childhood obesity and severe adult obesity. Nat Genet 2007; 39:724-6.
19. Frayling T M, Timpson N J, Weedon M N et al. A common variant in the FTO gene is associated with body mass index and predisposes to childhood and adult obesity. Science 2007; 316:889-94.
20. Scuteri A, Sanna S, Chen W M et al. Genome-wide association scan shows genetic variants in the FTO gene are associated with obesity-related traits. PLoS Genet 2007; 3:e115.
21. Saunders C L, Chiodini B D, Sham P et al. Meta-analysis of genome-wide linkage studies in BMI and obesity. Obesity (Silver Spring) 2007; 15:2263-75.
22. Chambers J C, Elliott P, Zabaneh D et al. Common genetic variation near MC4R is associated with waist circumference and insulin resistance. Nat Genet 2008; 40:716-8.
23. Ranadive S A, Vaisse C. Lessons from extreme human obesity: monogenic disorders. Endocrinol Metab Clin North Am 2008; 37:733-51, x.
24. Farooqi I S. Monogenic human obesity. Front Horm Res 2008; 36:1-11.
25. Still C D, Benotti P, Wood G C et al. Outcomes of preoperative weight loss in high-risk patients undergoing gastric bypass surgery. Arch Surg 2007; 142:994-8; discussion 999.
26. Wood G C, Still C D, Chu X et al. Association of chromosome 9p21 SNPs with cardiovascular phenotypes in morbid obesity using electronic health record data. Genomic Med 2008; 2:33-43.
27. McPherson R, Pertsemlidis A, Kavaslar N et al. A common allele on chromosome 9 associated with coronary heart disease. Science 2007; 316:1488-91.
28. Saxena R, Voight B F, Lyssenko V et al. Genome-wide association analysis identifies loci for type 2 diabetes and triglyceride levels. Science 2007; 316:1331-6.
29. Franks P W, Jablonski K A, Delahanty L M et al. Assessing gene-treatment interactions at the FTO and INSIG2 loci on obesity-related traits in the Diabetes Prevention Program. Diabetologia 2008; 51:2214-2223.
30. Celio A A, Wilfley D E, Crow S J, Mitchell J, Walsh B T. A comparison of the binge eating scale, questionnaire for eating and weight patterns-revised, and eating disorder examination questionnaire with instructions with the eating disorder examination in the assessment of binge eating disorder and its symptoms. Int J Eat Disord 2004; 36:434-44.
31. Chung W K, Leibel R L. Considerations regarding the genetics of obesity. Obesity (Silver Spring) 2008; 16 Suppl 3:S33-9.
32. Reinehr T, Hinney A, Nguyen T T, Hebebrand J. Evidence of an influence of a polymorphism near the INSIG2 on weight loss during a lifestyle intervention in obese children and adolescents. Diabetes 2008; 57:623-6.
33. Lappalainen T J, Tolppanen A M, Kolehmainen M et al. The Common Variant in the FTO Gene Did Not Modify the Effect of Lifestyle Changes on Body Weight: The Finnish Diabetes Prevention Study. Obesity (Silver Spring) 2009.
34. Haupt A, Thamer C, Maehann J et al. Impact of variation in the FTO gene on whole body fat distribution, ectopic fat, and weight loss. Obesity (Silver Spring) 2008; 16:1969-72.
35. Reinehr T, Hebebrand J, Friedel S et al. Lifestyle intervention in obese children with variations in the melanocortin 4 receptor gene. Obesity (Silver Spring) 2009; 17:382-9.
36. Sesti G, Perego L, Cardellini M et al. Impact of common polymorphisms in candidate genes for insulin resistance and obesity on weight loss of morbidly obese subjects after laparoscopic adjustable gastric banding and hypocaloric diet. J Clin Endocrinol Metab 2005; 90:5064-9.
37. Chen H H, Lee W J, Wang W, Huang M T, Lee Y C, Pan W H. Ala55Val polymorphism on UCP2 gene predicts greater weight loss in morbidly obese patients undergoing gastric banding. Obes Surg 2007; 17:926-33.
38. de Luis D A, Pacheco D, Aller R et al. Influence of −55CT Polymorphism of UCP3 Gene on Surgical Results of Biliopancreatic Diversion. Obes Surg 2008.
39. Potoczna N, Wertli M, Steffen R, Ricklin T, Lentes K U, Horber F F. G protein polymorphisms do not predict weight loss and improvement of hypertension in severely obese patients. J Gastrointest Surg 2004; 8:862-8; discussion 868.
40. Lubrano-Berthelier C, Dubern B, Lacorte J M et al. Melanocortin 4 receptor mutations in a large cohort of severely obese adults: prevalence, functional classification, genotype-phenotype relationship, and lack of association with binge eating. J Clin Endocrinol Metab 2006; 91:1811-8.
41. Haupt A, Thamer C, Staiger H et al. Variation in the FTO Gene Influences Food Intake but not Energy Expenditure. Exp Clin Endocrinol Diabetes 2008.
42. Cecil J E, Tavendale R, Watt P, Hetherington M M, Palmer C N. An obesity-associated FTO gene variant and increased energy intake in children. N Engl J Med 2008; 359:2558-66.
43. Andreasen C H, Stender-Petersen K L, Mogensen M S et al. Low physical activity accentuates the effect of the FTO rs9939609 polymorphism on body fat accumulation. Diabetes 2008; 57:95-101.
44. Branson R, Potoczna N, Kral J O, Lentes K U, Hoehe M R, Horber F F, Binge eating as a major phenotype of melanocortin 4 receptor gene mutations. N Engl J Med 2003; 348:1096-103.
45. Hebebrand J, Geller F, Dempfle A et al. Binge-eating episodes are not characteristic of carriers of melanocortin-4 receptor gene mutations. Mol Psychiatry 2004; 9:796-800.
46. Pajecki D, Dalcanalle L, Souza de Oliveira C P, et al. Follow-up of Roux-en-Y gastric bypass patients at 5 or more years postoperatively. Obes Surg. 2007; 17 (5):601-607.
47. Walley A J, Blakemore A I, Froguel P. Genetics of obesity and the prediction of risk for health. Hum Mol Genet, 2006; 15(2):R124-R130.
48. Ahituv N, Kavaslar N, Schackwitz W, et al. Medical sequencing at the extremes of human body mass. Am J Hum Genet. 2007; 80(4):779-791.
49. Hall D H, Rahman T, Avery P J, Keavney B. INSIG-2 promoter polymorphism and obesity related phenotypes: association study in 1428 members of 248 families. BMC Med Genet. 2006; 7:83.
50. Smith A J, Cooper J A, Li L K, Humphries S E. INSIG2 gene polymorphism is not associated with obesity in Caucasian, Afro-Caribbean and Indian subjects. Int J Obes (Lond). 2007; (11).
51. Dina C, Meyre D, Samson C, et al. Comment on “A common genetic variant is associated with adult and childhood obesity”. Science. 2007; 315(5809):187.
52. Loos R J, Barroso I, O'Rahilly S, Wareham N J. Comment on “A common genetic variant is associated with adult and childhood obesity”. Science. 2007; 315 (5809):187.
53. Rosskopf D, Bornhorst A, Rimmbach C, et al. Comment on “A common genetic variant is associated with adult and childhood obesity”. Science. 2007; 315 (5809):187.
54. Kumar J, Sunkishala R R, Karthikeyan G, Sengupta S. The common genetic variant upstream of INSIG2 gene is not associated with obesity in Indian population. Clin Genet, 2007; 71(5):415-418.
55. Field S F, Howson J M, Walker N M, Dunger D B, Todd J A. Analysis of the obesity gene FTO in 14,803 type 1 diabetes cases and controls. Diabetologia. 2007; 50(10):2218-2220.
56. Dahlman I, Amer P. Obesity and polymorphisms in genes regulating human adipose tissue. Int J Obes (Load). 2007; 31(10:1629-1141.
57. Bell C G, Meyre D, Petretto E, et al. No contribution of angiotensin-converting enzyme (ACE) gene variants to severe obesity: a model for comprehensive case/control and quantitative cladistic analysis of ACE in human diseases. Eur J Hum Genet. 2007; 15(3):320-327.
58. Swarbrick M M, Waldenmaier B, Pennacchio L A, et al. Lack of support for the association between GAD2 polymorphisms and severe human obesity. PLoS Biol. 2005; 3(9):e315.
59. Talmud P J, Palmen J, Wolf A M, Beisiegel U. Investigation into the role of the hormone sensitive lipase −60C_G promoter variant in morbid obesity. Nutr Metab Cardiovasc Dis. 2005; 15(1):31-35.
60. Hensrud D D, Klein S. Extreme obesity: a new medical crisis in the United States. Mayo Clin Proc. 2006; 81(10)(suppl):S5-S10.
61. Frazer K A, Ballinger D G, Cox D R, et al. A second generation human haplotype map of over 3.1 million SNPs. Nature. 2007; 449(7164):851-861.
62. Gong Y, Lee J N, Brown M S, Goldstein J L, Ye J. Juxtamembranous aspartic acid in Insig-1 and Insig-2 is required for cholesterol homeostasis. Proc Natl Acad Sci U S A. 2006; 103(16):6154-6159.
63. Peters T, Ausmeier K, Ruther U. Cloning of Fatso (Fto), a novel gene deleted by the Fused toes (Ft) mouse mutation. Mamm Genome, 1999; 10(10):983-986.
64. Anselme I, Laclef C, Lanaud M, Ruther U, Schneider-Maunoury S. Defects in brain patterning and head morphogenesis in the mouse mutant Fused toes. Dev Biol. 2007; 304(1):208-220,
65. Pope G D, Finlayson S R, Kemp J A, Birkmeyer J D. Life expectancy benefits of gastric bypass surgery. Surg Innov. 2006; 13(4):265-273.
66. Potoczna N, Branson R, Kral J G, et al. Gene variants and binge eating as predictors of comorbidity and outcome of treatment in severe obesity. J Gastrointest Surg. 2004; (8):971-982.
67. Swen J J, Huizing a T W, Gelderblom H, et al. Translating pharmacogenomics: challenges on the road to the clinic. PLoS Med. 2007; 4(8):e209.

What is claimed is:

1. A method for determining a patient's resistance to weight loss, comprising:
obtaining a sample from a patient containing nucleic acid;
isolating the nucleic acid from said sample;
determining the residue present at single nucleotide polymorphism identified by identification numbers rs7566605, rs9939609, rs17782313 and rs6235 for each allele of the single nucleotide polymorphism; and
obtaining a total number of obesity alleles by summing the number of times a C residue is present at rs7566605, an A residue is present at rs9939609, a C residue is present at rs17782313 and a C residue is present at rs6235;
wherein, if the total number of obesity alleles is from 5 to 8, the patient is determined as being resistive to weight loss.

2. The method of claim 1, wherein the sample is a bodily fluid sample.

3. The method of claim 2, wherein the bodily fluid sample is blood.

4. The method of claim 1, wherein the sample is a tissue sample.

5. A method for determining a suitable type of bariatric surgery to treat a patient, comprising:
obtaining a sample from a patient containing nucleic acid;
isolating the nucleic acid from said sample;
determining the residue present at single nucleotide polymorphism identified by identification numbers rs7566605, rs9939609, rs17782313 and rs6235 for each allele of the single nucleotide polymorphism; and
obtaining a total number of obesity alleles by summing the number of times a C residue is present at rs7566605, an A residue is present at rs9939609, a C residue is present at rs17782313 and a C residue is present at rs6235;
wherein, if the total number of obesity alleles is from 0 to 4, the patient is determined as being suitable for standard bariatric surgery; and wherein if the total number of obesity alleles is from 5 to 8, the patient is determined as being suitable for a highly malabsorptive procedure.

6. A method for determining a patient's resistance to weight loss, comprising:
obtaining a sample from a patient containing nucleic acid;
isolating the nucleic acid from said sample;
determining the residue present at a single nucleotide polymorphism identified by identification numbers rs7566605, rs9939609, rs17782313 and rs6235 for each allele of the single nucleotide polymorphism; and
obtaining a total number of homozygous obese genotypes by summing the number of times both of the patient's alleles have a C residue at rs7566605, both of the patient's alleles have an A residue at rs9939609, both of the patient's alleles have a C residue at rs17782313 and both of the patient's alleles have a C residue at rs6235;
wherein, if the total number of homozygous obese genotypes is from 2 to 4, the patient is determined as being resistive to weight loss.

7. A method for determining a patient's resistance to weight loss, comprising:
obtaining a sample from a patient containing nucleic acid;
isolating the nucleic acid from said sample;
determining the residue present at one or more single nucleotide polymorphism selected from the group consisting of single nucleotide polymorphism identified by identification numbers rs7566605, rs9939609, rs17782313 and rs6235 for each allele of the single nucleotide polymorphism; and
ascertaining whether the single nucleotide polymorphism cluster shows a homozygous obese genotype by determining whether both of the patient's alleles have a C residue at rs7566605, both of the patient's alleles have an A residue at rs9939609, both of the patient's alleles have a C residue at rs17782313 or both of the patient's alleles have a C residue at rs6235;
wherein, if the patient has a homozygous obese genotype at the selected cluster, the patient is determined as being resistive to weight loss.

8. A method for determining a patient's resistance to weight loss, comprising:
obtaining a sample from a patient containing nucleic acid;
isolating the nucleic acid from said sample;
determining the sequence of a nucleic acid comprising SEQ ID NO: 1, the sequence of a nucleic acid comprising SEQ ID NO: 2, the sequence of a nucleic acid comprising SEQ ID NO: 3, and the sequence of a nucleic acid comprising SEQ ID NO: 4; and
obtaining a total number of obesity alleles by summing the number of times a C residue is present at position 11 of SEQ ID NO: 1, an A residue is present at position 11 of SEQ ID NO:2, a C residue is present at position 11 of SEQ ID NO:3 and a C residue is present at position 11 of SEQ ID NO:4;
wherein, if the total number of obesity alleles is from 5 to 8, the patient is determined as being resistive to weight loss.

9. The method of claim 8, wherein the sample is a bodily fluid sample.

10. The method of claim 9, wherein the bodily fluid sample is blood.

11. The method of claim 8, wherein the sample is a tissue sample.

12. A method for determining a patient's susceptibility to binge eating, comprising:
obtaining a sample from a patient containing nucleic acid;
isolating the nucleic acid from said sample;
determining the residue present at a single nucleotide polymorphism identified by identification numbers rs7566605 for each allele of the single nucleotide polymorphism; and
ascertaining whether the single nucleotide polymorphism shows a homozygous obese genotype by determining whether both of the patient's alleles have a C residue at rs7566605;
wherein, if the patient has a homozygous obese genotype at the selected cluster, the patient is determined as being susceptible to binge eating.

13. A method for determining a patient's metabolic rate or resting energy expenditure or oxygen consumption (VO₂), comprising:
obtaining a sample from a patient containing nucleic acid;
isolating the nucleic acid from said sample;
determining the residue present at a single nucleotide polymorphism identified by identification numbers rs7566605 for each allele of the single nucleotide polymorphism; and
ascertaining whether the single nucleotide polymorphism shows a homozygous obese genotype by determining whether both of the patient's alleles have a C residue at rs7566605;
wherein, if the patient has a homozygous obese genotype at the selected cluster, the patient is determined as being susceptible to having a low metabolic rate or resting energy expenditure or oxygen consumption (VO₂).
